(19) United States
(12) Patent Application Publication
(10) Pub. No.: US 2025/0261912 A1
(43) Pub. Date: Aug. 21, 2025

(54) INTRAOPERATIVE NEURAL MONITORING SYSTEM AND METHOD

(71) Applicant: DePuy Synthes Products, Inc.

(72) Inventors: Christopher Wybo, Brighton, MI (US); Darren Scarfe, LaSalle (CA); Aashish Shah, Ann Arbor, MI (US); Jacob A. Marks, Mansfield, MA (US); James G. Lee, South Weymouth, MA (US)

(21) Appl. No.: 19/189,774

(22) Filed: Apr. 25, 2025

Related U.S. Application Data
(63) Continuation of application No. 17/878,705, filed on Aug. 1, 2022, now Pat. No. 12,303,301.
Application No. 17/878,705 is a continuation of application No. 16/586,121, filed on Sep. 27, 2019, now Pat. No. 11,399,777.

(51) Int. Cl. A61B 5/00; A61B 5/11; G06T 11/20

(52) U.S. Cl. CPC A61B 5/743; A61B 5/1104; A61B 5/1107; A61B 5/4893; G06T 11/206; A61B 2562/0219

(57) ABSTRACT

A neuromuscular sensing method includes transmitting an electrical stimulus from a distal end portion of an elongate stimulator extending within an intracorporeal treatment area of a subject and detecting an artificially induced response of at least two muscles of the subject in response to the transmitted electrical stimulus, each response being detected by a respective sensor in mechanical communication with the muscle. A graphical user interface (GUI) is transitioned from a first state to a second state; the first state indicating to the transmission of a stimulus with no artificially induced response detected, and the second state indicating the detection of the artificially induced response of the at least two muscles, and further identifying one of the at least two muscles as a primary muscle response.

### CROSS REFERENCE TO RELATED APPLICATIONS

This application is a continuation of U.S. patent application Ser. No. 17/878,705 (now U.S. Pat. No. ______), which is a continuation of U.S. Patent Application No. 16/586, 121 (now U.S. Pat. No. 11,399,777), the disclosures of each of which are incorporated by reference herein in their entireties.

### TECHNICAL FIELD

The present disclosure relates generally to a sensing device used with a surgical diagnostic system for detecting artificially induced neuromuscular activity.

### BACKGROUND

Traditional surgical practices emphasize the importance of recognizing or verifying the location of nerves to avoid injuring them. Advances in surgical techniques include development of techniques including ever smaller exposures, such as minimally invasive surgical procedures, and the insertion of ever more complex medical devices.

With these advances in surgical techniques, there is a corresponding need for improvements in methods of detecting and/or avoiding nerves.

### SUMMARY

Provided herein is an intraoperative neural monitoring system, and a neuromuscular sensing method that is operative to promptly alert a user to the occurrence of an artificially induced muscle response during a surgical procedure. Such a response is indicative of the presence of a nerve within an intracorporeal treatment area and would suggest caution to the user/surgeon. Conversely, a lack of a response is also informative and would suggest no nerve in the immediate area.

An embodiment of the method includes transmitting an electrical stimulus from a distal end portion of an elongate stimulator extending within an intracorporeal treatment area of a subject, and detecting an artificially induced response of at least two muscles of the subject in response to the transmitted electrical stimulus, each response being detected by a respective sensor in mechanical communication with the muscle. Following the detection, a graphical user interface (GUI) is transitioned from a first state to a second state. The first state indicates the transmission of a stimulus with no artificially induced response detected, and the second state indicates the detection of the artificially induced response of the at least two muscles. The second state further identifies one of the at least two muscles as providing the primary muscle response.

An embodiment of the intraoperative neural monitoring system includes a plurality of mechanical sensors, an elongate stimulator, and a host system. Each of the plurality of mechanical sensors is operative to monitor a different muscle of a subject.

The elongate stimulator is operative to extend within an intracorporeal treatment area of a subject and to transmit an electrical stimulus from a distal end portion, and the host system is in communication with the plurality of mechanical sensors.

The host system includes a display and a processor. The processor is configured to receive an output signal from each of the mechanical sensors, where the output signal corresponding to the movement of the monitored muscles of the subject. It is further configured to display a graphical user interface (GUI) via the display, and detect an artificially induced response of at least two muscles of the subject in response to the electrical stimulus. The processor is configured to transition the GUI from a first state to a second state in response to the detected artificially induced response of the at least two muscles. The first state indicates the transmission of a stimulus with no artificially induced response detected, and the second state indicates the detection of the artificially induced response of the at least two muscles, while further identifying one of the at least two muscles as a primary muscle response.

The features and advantages and other features and advantages of the present technology are readily apparent from the following detailed description when taken in connection with the accompanying drawings.

“A,” “an,” “the,” “at least one,” and “one or more” are used interchangeably to indicate that at least one of the item is present; a plurality of such items may be present unless the context clearly indicates otherwise. All numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range. Each value within a range and the endpoints of a range are hereby all disclosed as separate embodiment.

### BRIEF DESCRIPTION OF THE DRAWINGS

FIG. 1 is a schematic diagram of a neural monitoring system, including a sleeve-based sensing device, for detecting an artificially-induced mechanical muscle response.

FIG. 2 is an enlarged schematic anterior view of a sleeve-based sensing device provided on a leg of a subject.

FIG. 3 is a schematic illustration of a display presenting a graphical user interface (GUI) that identifies an intraoperative neural monitoring system as being in a standby state.

FIG. 4 is a schematic block diagram of a pre-operative setup method for an intraoperative neural monitoring system.

FIG. 5 is a schematic illustration of a display presenting a GUI that identifies an intraoperative neural monitoring system as stimulating without any detected muscle response.

FIG. 6 is a schematic illustration of a display presenting a GUI that identifies an intraoperative neural monitoring system as stimulating and detecting an artificially induced muscle response.

FIG. 7 is a schematic illustration of a display presenting a GUI that identifies an intraoperative neural monitoring system as stimulating and detecting a spontaneous muscle response.

### DETAILED DESCRIPTION

The present disclosure provides a system that enables a surgeon/user to rapidly identify the presence of nerves within a surgical area internal to a subject. Such a system may be useful, for example, during spinal surgery, where there is a myriad of nerve bundles exiting the spine and extending through adjacent muscle tissue. The present technology relies on understanding that when a nerve is depolarized, the muscles innervated by that nerve contract. The present system is operative to monitor certain muscle groups of the subject and discriminate between muscle motion that is artificially induced by a transmitted electrical stimulus, muscle motion that is artificially induced, though not by a transmitted electrical stimulus (i.e., a “spontaneous response” which may be caused, for example, by mechanical strain), or other intended or environmental motion. If the system detects an artificially induced response to a transmitted stimulus, or a spontaneous response, it may transition a graphical user interface from a generic “stimulating” state to a more specific detection state. The detection state may identify the type of response, as well as all muscles that exhibit motion characteristic of that type of response. Furthermore, the system may alert the surgeon to which muscle is presenting the primary response to the depolarized nerve, and which are presenting ancillary responses (with the understanding that one nerve root may innervate multiple muscle groups to differing degrees).

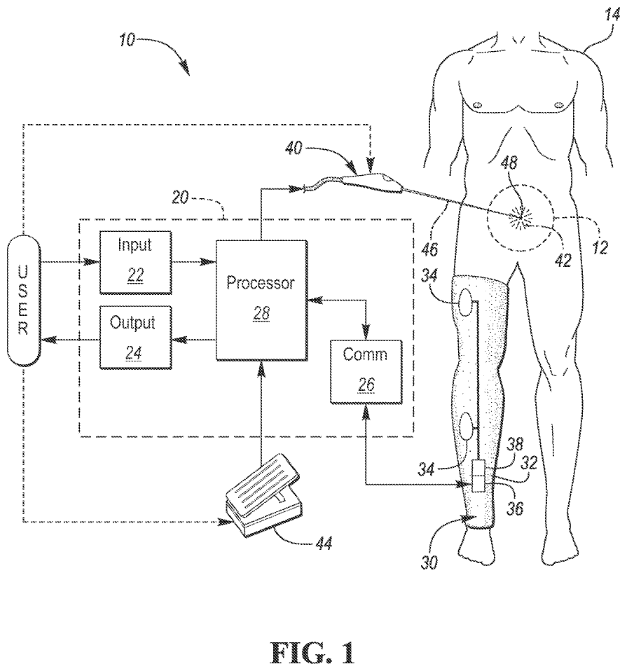

Referring to the drawings, wherein like reference numerals are used to identify like or identical components in the various views, FIG. 1 schematically illustrates a neural monitoring system 10 that may be used to identify the presence of one or more nerves within an intracorporeal treatment area 12 of a subject 14, such as during a surgical procedure. As will be described in greater detail below, the system 10 may monitor one or more muscles of the subject 14 for a neuromuscular response that is indicative of a stimulus-induced depolarization of a nerve (i.e., an artificially induced neuromuscular response). If a response of the muscle to the stimulus is detected during the procedure, the system 10 may provide an alert or indication to the surgeon, which may enable the surgeon to take an appropriate action if such action is warranted.

As used herein, an “artificially induced neuromuscular response” is a response of a muscle to a depolarizing stimulus that is applied to a nerve innervating the muscle. In general, the response is “artificially induced” because the nerve is depolarized directly by the stimulus, instead of, for example, the stimulus being received through an intermediate sensory means (e.g., sight, sound, taste, smell, and touch). An example of a stimulus that may cause an “artificially-induced” muscle response may include an electrical current applied directly to the nerve or to intracorporeal tissue or fluid immediately surrounding the nerve. In such an example, if the applied electrical current is sufficiently strong and/or sufficiently close to the nerve, it may artificially cause the nerve to depolarize (resulting in a corresponding contraction of the muscle or muscles innervated by that nerve).

During an artificially induced neuromuscular response, a muscle innervated by the artificially depolarized nerve may physically contract or relax (i.e., a mechanical response) and/or the electrical potential throughout the muscle may be altered. Mechanical responses may primarily occur along a longitudinal direction of the muscle (i.e., a direction aligned with the constituent fibers of the muscle), though may further result in a respective swelling/relaxing of the muscle in a lateral direction (which may be substantially normal to the skin for most skeletal muscles). This local movement of the muscle during an artificially-induced mechanical muscle response may be measured relative to the position of the muscle when in a non-stimulated state.

The neural monitoring system IO may generally include a host system 20 and a sensing device 30 that may cooperate to detect a neuromuscular response of a muscle to a stimulus 42 provided by a stimulator 40. As schematically shown in FIG. 1, the host system 20 may include one or more input devices 22 that are operative to receive information from the surgeon, one or more output devices 24 that are operative to communicate alerts or to provide informational feedback to the surgeon, communication circuitry 26 operative to communicate with the sensing device 30, and a processor 28 that is operative to at least manage the flow of information between the input devices 22, output devices 24 and communication circuitry 26.

In general, the one or more input devices 22 may include a keyboard, a mouse, and/or a digitizer provided with a touch-screen display. These devices may receive pre-operative case information or may permit a surgeon to alter various intraoperative parameters, alarm limits, or other case information before or during a procedure. In some embodiments, the stimulator 40 and/or a foot pedal 44 may provide additional input to the host system 20. This input may be in the form of an analog or digital signal that is indicative of the delivery and/or magnitude of a stimulus. The output device 24 may include, for example, a visual display such as an LED/LCD display 100 (further illustrated in FIG. 3), one or more indicator lights, or speakers capable of providing an audible alert to the surgeon.

The sensing device 30 is the portion of the system 10 that directly contacts the subject 14 and is responsible for, at a minimum, sensing/detecting neuromuscular responses of the subject 14. The sensing device 30 may include a carrier material 32 that is operative to be secured to the subject 14, and a plurality of neuromuscular sensors 34 that are coupled with the carrier material 32 and each operative to monitor a neuromuscular response of a different muscle group of the subject 14. The sensing device 30 may further include communication circuitry 36 that is operative to digitally communicate with the communication circuitry 26 of the host system 20, and a local processor 38 that is in communication with the plurality of neuromuscular sensors 34 and with the communication circuitry 36. In general, processors used with the present system 10 (e.g., processors 28, 38) may each be embodied as one or multiple digital computers, data processing devices, and/or digital signal processors (DSPs), which may have one or more microcontrollers or central processing units (CPUs), read only memory (ROM), random access memory (RAM), electrically-erasable programmable read only memory (EEPROM), a high-speed clock, analog-to-digital (AID) circuitry, digital-to-analog (D/A) circuitry, input/output (I/O) circuitry, and/or signal conditioning and buffering electronics.

While the specific nature of the sensing device 30 and carrier material 32 may vary based on the location of the surgical site and nature of the surgical procedure, in some embodiments, the carrier material 32 may resemble a cuff or sleeve that is secured around a limb of the subject. Such a design may be suitable, for example, with a spinal procedure where nerves existing within the surgical site are known to innervate peripheral muscles of the arms or legs.

In some embodiments, the carrier material 32 may be a separate therapeutic or diagnostic device that is already common in surgical applications. For example, in a spinal procedure involving one or more of the L2-S 1 vertebrae, it is known that nerve roots innervating the leg muscles may lie within the surgical area. During such procedures, however, compression-type anti-embolism stockings (Thrombo-Embolic-Deterrent (“TED”) hose) are typically provided around a subject's legs and feet to discourage blood clot formation. Thus, in one embodiment the carrier material 32 may be an elastic sleeve/stocking configured to apply a compressive force to the subject's leg when worn, thus eliminating the need for separate TED hose. Such a compression against the subject may present itself as an elastic tension/strain in the carrier material itself (also referred to as a “tension fit”). In surgical procedures performed higher on the spine, the carrier material 32 may include, for example, a blood pressure cuff worn around the subject's arm (or else may include functionality similar to that of a standard blood pressure cuff). In these examples, the carrier material 32 serves a function outside of that of a dedicated neuromuscular sensing device, and thus provides efficiencies in pre-op preparation and planning, while also allowing monitoring access on sometimes crowded limbs.

In still other embodiments, the carrier material 32 may be a self-adhesive patch that is separate for each sensor. From the perspective of the sensing device 30, the carrier material's main purpose is to hold the neuromuscular sensors 34 in relatively stable contact with the skin of the subject.

While in one embodiment, the neuromuscular sensors 34 can be or can include EMG electrodes, in a more preferred embodiment, the neuromuscular sensors 34 each include a respective mechanical sensor that is operative to monitor a mechanical response of a directly adjacent muscle group. In various embodiments, the mechanical sensor in each neuromuscular sensor 34 may include, for example, a strain gauge, a pressure/force transducer, a position encoder, an accelerometer, a piezoelectric material, or any other transducer or combination of transducers that may convert a physical motion into a variable electrical signal.

As noted above, the system 10 may further include one or more elongate medical instruments 40 (i.e., stimulators 40) that are capable of selectively providing a stimulus 42 within the intracorporeal treatment area 12 of the subject 14. For example, in one configuration, the elongate medical instrument 40 may include a probe 46 (e.g., a ball-tip probe, k-wire, or needle) that has an electrode 48 disposed on a distal end portion. The electrode 48 may be selectively electrified, at either the request of a user/physician, or at the command of the processor 28, to provide an electrical stimulus 42 to intracorporeal tissue of the subject. In other configurations, the elongate medical instrument 40 may include a dilator, retractor, clip, cautery probe, pedicle screw, or any other medical instrument that may be used in an invasive medical procedure. Regardless of the instrument, if the intended artificial stimulus is an electrical current, the instrument 40 may include a selectively electrifiable electrode 48 disposed at a portion of the instrument that is intended to contact tissue within the intracorporeal treatment area 12 during the procedure.

During a surgical procedure, the user/surgeon may selectively administer the stimulus to intracorporeal tissue within the treatment area 12 to identify the presence of one or more nerve bundles or fibers. For an electrical stimulus 42, the user/surgeon may administer the stimulus, for example, upon depressing a button or foot pedal 44 that is in communication with the host system 20. The electrical stimulus 42 may, for example, be a periodic stimulus that includes a plurality of sequential discrete pulses (e.g., a step pulse) provided at a frequency of less than about 10 Hz, or from about 1 Hz to about 5 Hz, and preferably between about 2 Hz and about 4 Hz. Each pulse may have a pulse width within the range of about 50 μs to about 400 μs. In other examples, the discrete pulse may have a pulse width within the range of about 50 μs to about 200 μs, or within the range of about 75 μs to about 125 μs. Additionally, in some embodiments, the current amplitude of each pulse may be independently controllable.

If a nerve extends within a predetermined distance of the electrode 48, the electrical stimulus 42 may cause the nerve to depolarize, resulting in a mechanical twitch of a muscle that is innervated by the nerve (i.e., an artificially-induced mechanical muscle response). In general, the magnitude of the response/twitch may be directly correlated to the distance between the electrode and the nerve, the impedance between the electrical stimulus and the ground patch, and the magnitude of the stimulus current. In one configuration, a lookup table may be employed by the processor 28 to provide an approximate distance between the electrode and the nerve, given a known stimulus magnitude and a measured mechanical muscle response.

As noted above, each mechanical sensor may be specially configured to monitor a local mechanical movement of an adjacent muscle group of the subject 14. In response to this sensed movement, each respective mechanical sensor may generate a mechanomyography (MMG) output signal that corresponds to the sensed mechanical movement, force, and/or response of the adjacent muscle. The MMG output signal may be either a digital or analog signal, and the sensor 34 may further include any communication circuitry or local processing circuitry that may be required to transmit the MMG output signal (or a suitable representation thereof) to the processor 38 via a wired or wireless communications.

The communications circuitry of the sensing device 30 may digitally communicate with the communications circuitry 26 of the host system 20 through any suitable wired or wireless communications means. The respective circuitry 26, 36 may permit unidirectional or bidirectional communications, and may be chosen, in part, according to the number of sensing devices 30 are concurrently used during a procedure (noting that many Bluetooth protocols require point-to-point pairing). Suitable wired protocols include I2C, CAN, TCP/IP, while suitable wireless protocols include IEEE 802.11, Bluetooth, ZigBee, NFC, RFID or the like.

The following description of setup and operation of the sensing device 30 will be made with respect to a lumbar spinal procedure where the sensing device 30 is provided on a leg of the subject 14. It should be appreciated that the specific nature procedure is illustrative, and should not be read as limiting. The following principles of operation can similarly apply to other procedures, such as spinal procedures performed on the cervical spine (e.g., CS-TI-with the sensing device provided on an arm of the subject), or with other such procedures.

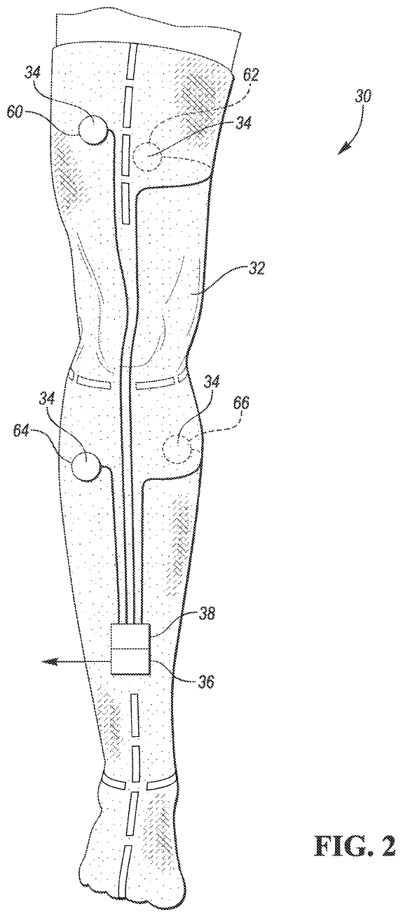

FIG. 2 schematically illustrates one embodiment of a sensing device 30 that may be used during the performance of a surgical procedure on the lumbar spine. As shown, in this embodiment, the carrier material 32 may be a sleeve that is operative to maintain an elastic tension fit about a leg of the subject. The sensing device 30 includes a plurality of neuromuscular sensors 34 embedded within, or otherwise directly coupled to the carrier material 32. Each sensor 34 is positioned on the carrier material 32 such that it may monitor a response of a different muscle group of the leg. For example, a first sensor 60 may be located on an anterior portion of the thigh, such that it approximately centered above/outward of the vastus lateralis or vastus medialis muscle when the sleeve is properly positioned on the leg of the subject. A second sensor 62 may be located on a posterior portion of the thigh, such that it approximately centered above/outward of the bicepsfemoris muscle when the sleeve is properly positioned on the leg of the subject. A third sensor 64 may be located on an anterolateral portion of the lower leg, such that it isapproximately centered above/outward of the tibialis anterior muscle when the sleeve is properly positioned on the leg of the subject. Finally, with some procedures lower on the spine, a fourth sensor 66 may be located on the posterior portion of the lower leg, such that it is approximately centered above/outward of the gastrocnemius muscle when the sleeve is properly positioned on the leg of the subject.

During a surgical procedure, the surgeon/user may be presented with a visual indication of the mechanical response detected by each neuromuscular sensor 34 via an LED/LCD display 100 that is associated with the host system 20. FIG. 3 provides one illustration of an informational graphical user interface (GUI) 102 that may be used to present such information. As discussed below, the GUI 102 may have unique characteristics that enable the surgeon to quickly recognize the state of the system, determine if a nerve is proximate to the stimulator, and see the output signal from one or more sensors. Additionally, if multiple sensors are alerting, the user may quickly assess which sensor channel represents a primary response, and which sensor channels are alerting in an ancillary manner.

As shown in FIG. 3, the GUI 102 may include a plurality of icons/display elements 104, with each respective display element 104 representing a different potential sensor channel that could be used within a surgical procedure. The number of display elements 104 should preferably be greater than or equal to the number of sensors 34 in mechanical communication with the subject 14 and/or greater than or equal to the number of muscle groups actively monitored by the sensing device 30.

In some embodiments, the number of display elements 104 may correspond to the number of sensors 34 that are supported by the system. For example, if the system is capable of eight sensing channels, then the GUI 102 may display eight icons 104, each corresponding to a different available sensing channel.

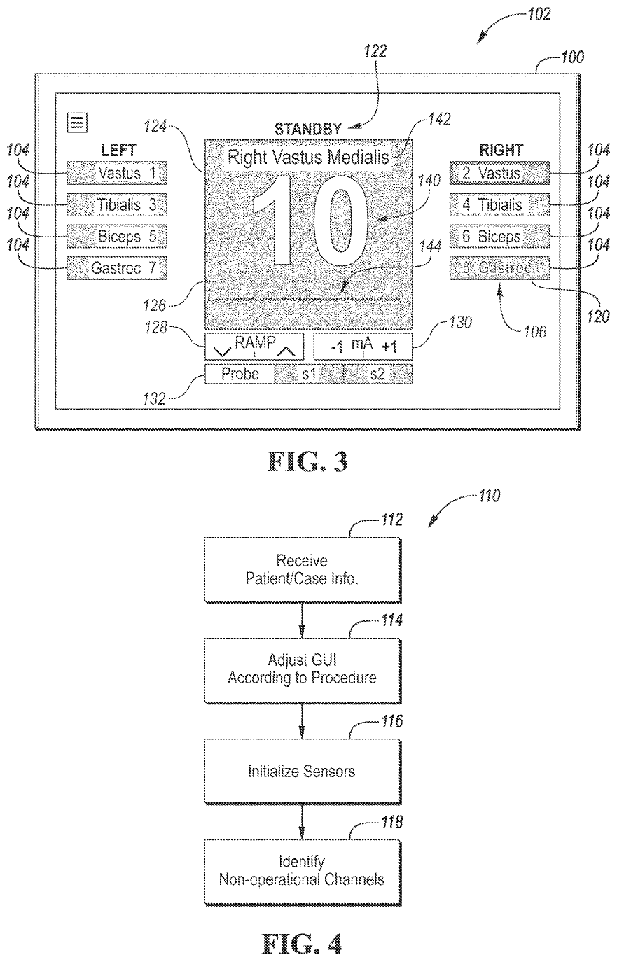

In another embodiment, the number, name, and/or arrangement of each of the plurality of icons 104 may be actively modified according to the nature of the surgery and/or from an actual initialization routine. FIG. 4 schematically represents one embodiment of a pre-op configuration method 110 for preparing the GUI 102 for a surgical procedure. As shown, the method 110 begins at 112 with the user entering patient and/or case-specific information. Using this information, the host system 20 may structure the GUI 102 to include (at 114) a number and arrangement of display elements 104 that corresponds to the nature of the procedure. For example, as shown in FIG. 3, the nature of the procedure may have been entered as a lumbar interbody fusion (LIF) of the L3-L4 spinal vertebrae. From this, the host system 20 may recognize that the implicated nerve roots, and the nerve roots exiting adjacent foramen innervate the primary muscles of the leg (vastus, tibialis, biceps, and gastroc). Therefore, as shown in FIG. 3, the GUI 102 may include display elements 104 corresponding to these respective muscle groups.

In one configuration, the GUI 102 may include the pre-determined, procedure-specific display elements 104 regardless of whether a corresponding sensor is connected. In such an embodiment, the existence of the various display elements may serve as a reminder to affix and initialize the various sensors. In another configuration, the GUI 102 may only include a display element 104 if the corresponding sensor is attached. For example, in the example shown in FIG. 3, if the surgeon was confident that the procedure was limited to the left side, he may choose to omit sensor placement on the right side. In such an embodiment, the display elements corresponding to the muscles of the subject's right leg (i.e., elements 106, collectively) may be hidden from view either by the surgeon or automatically by the system.

With continued reference to FIG. 3, in addition to the display elements 104 corresponding to the various sensors/sensor channels, the GUI 102 may include a central status window 126, a ramp input element 128 for initiating a gradual increase or decrease in the stimulus current over time, a current control element 130 for causing a discrete change in the stimulus current (e.g., in a 0.5 mA or 1 mA increment), and a probe selector element 132 for changing which stimulator 40 (in FIG. 1) is being controlled by elements 128, 130 if more than one stimulator is being used.

The central status window 126 may then include an indication 140 of the stimulus intensity, while also providing the user with the ability to selectively monitor one of the sensor channels. In doing so, the central status window 126 may include a textual indicator 142 that expresses which channel is being monitored (i.e., the “displayed channel”), along with a graph element 144 (better shown in FIG. 6) that graphically represents the real-time MMG output signal from the selected sensor. During use, the displayed channel may be manually selected by tapping on a respective sensor display element 104. For example, as shown in FIG. 3, the user has selected the Right Vastus channel, which is reflected, both by a change in the appearance of the respective display element 104, as well as textually at 142.

Once the sensors 34 are positioned on the subject 14, the host system 20 may then perform an initialization routine (at 116 in FIG. 4) to ensure that each sensor is operational and coordinated to the correct display element 104. In an embodiment, the initialization routine 116 may instruct the user, via the GUI 102, to tap on each sensor in a sequential manner. For example, it may prompt the user to first tap on the left vastus sensor. It may then listen for one sensor channel to transmit an MMG output signal corresponding to an external tap. If multiple sensor channels report a tap-like signal, the host system 20 may report an error or else re-prompt the user via the GUI 102. Once one single channel is identified in response to the prompt, the system 20 may then prompt the user to tap on a different sensor (e.g., left tibialis). This process may continue until each display element 104 is associated with a single sensor response. This manner of sensor initialization and coordination both ensures that there is an operational and responding sensor on each respective muscle group, while also relying on the user's expertise and anatomical knowledge to ensure proper sensor association to the various display elements 104.

While the tap-based manner of sensor initialization is a valuable technique for ensuring that each sensor is operational, other techniques are available for coordinating the various sensors 34 with the display elements 104. In such embodiments, however, the user feedback can also be used to provide a confirmation that the system's understanding of sensor placement and coordination with the display elements 104 is correct. It should be recognized, however that physical user confirmation, such as by tapping, is not strictly required. In some embodiments, sensors may be deemed operational solely by the presence of a transmitted MMG output signal.

In one embodiment, the sensors 34 may each have a unique network address or identifier that is used to preface communications with the host system. In a system where the sensors are provided in a predefined array, such as on an elastic sleeve or cuff, the address may convey the relative location of the sensor within the array. In some embodiments where the array is keyed to the user's body (e.g., including a feature that aids in alignment such as a glove, sock, or indicia that is intended to be anatomically aligned), the address may convey an absolute position of the sensor or a primary muscle group that is being sensed.

In still another embodiment, the host system 20 may make a preliminary estimation of sensor-muscle correspondence by analyzing how the sensor is oriented on the body (e.g., via an accelerometer that can sense a gravity vector). Such a technique is described in Ser. No. 16/158,734, filed on 12 Oct. 2018 (the '734 Application), which is incorporated by reference in its entirety. As described in the '734 Application, general orientation of the sensing device 30 may be determined in a global coordinate frame by examining a static component of one or more MMG output signals, i.e., where the static component is representative of the force of gravity. Given a patient's known posture, the gravity heading will inform the system about which side of the limb the sensor is positioned on. This understanding, together with the knowledge of where various sensors are located across the array/device 30 may allow the system to infer muscle correspondence. In addition to estimating the muscle correspondence, the system may also determine which side of the body the sensing device is positioned on by examining the gravity vector heading on sensors placed on asymmetric portions of the body, such as further discussed in the '734 Application. In one configuration, this concept of gravity-informed sensor coordination may be enhanced by prompts to the user, for example, to lift a particular limb, where distal sensors would have greater travel and rates of acceleration than proximal sensors.

Referring again to FIG. 4, once the operational status and correspondence of the sensors 34 with the display elements 104 is established (at 116), the GUI 102 may then reflect any sensors that are not operational (at 118), such as by highlighting the corresponding display element (e.g., display element 120 in FIG. 3) with an indicia (e.g., a red “X”) or by coloring it a different color (represented by the stippling pattern). Following this, the GUI 102 may convey the system's operational status (at 118) for example, to reflect that the system is set up and ready to stimulate, though presently in a standby state. As shown in FIG. 3, the system status (e.g., in standby) may be reflected by a textual status indicator 122 and/or by a visual indicator 124 such as the color of a portion of a portion of the screen.

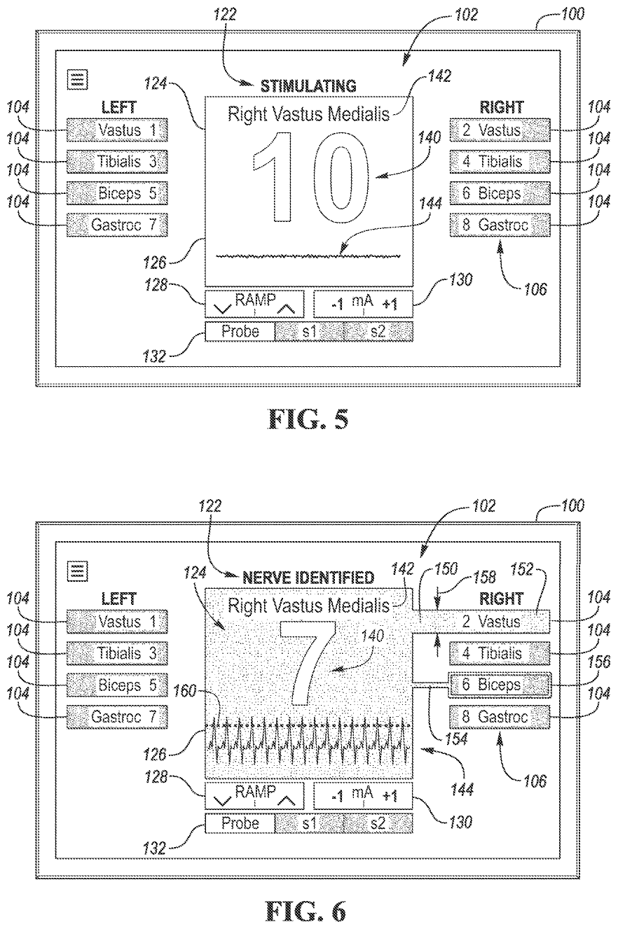

Referring to FIG. 5, once the stimulus beings to be administered, the GUI 102 may be altered to reflect that the system is now in a detection mode. For example, the textual status indicator 122 may reflect “stimulating” and/or a visual indicator 124, such as the color of the background of the central status window 126, may be altered to reflect the stimulating state. In one embodiment, a “standby” state may be reflected, for example, by a first color (e.g., gray), while the “stimulating” state may be reflected, for example, by a second color (e.g., green).

In one embodiment, the system may determine that the stimulator 40 is actively stimulating, for example, by referencing the commanded state (i.e., commanding a power transistor to conduct), however, it may be more informative to sense (e.g. via a sense resistor, current shunt, or inductive current sensor) that the stimulus is actually being administered to the subject. Such sensing may occur at or proximate to one or both of the probe or a ground-return patch that is attached to the subject's body. In this manner, the system may know not just that electrical current is being commanded, but that it is actually being transmitted to/through the subject's body. A loss of good contact between the stimulator and the subject may compromise the validity of the results, however, this may be detected by active monitoring.

Following the administering of the stimulus, the system may monitor the output from the various sensors to determine if there is an evoked muscle response sensed by any of the active sensors. The determination of an evoked muscle response may occur in the time domain, such as described, for example, in U.S. Pat. Nos. 8,343,079 and 8,343,065, which are incorporated by reference in their entirety, in the frequency domain, such as described in U.S. Patent Publication Nos. 2015/0051506 and 2015/0051507, which are incorporated by reference in their entirety, or through the use of pattern recognition techniques, neural networks, or other machine learning algorithms such as described, for example, in U.S. patent application Ser. No. 15/995,879. Analog detection techniques may, for example, compare the time derivative of acceleration of a muscle response to a threshold (i.e., where artificially induced responses have greater da/dt values than natural or externally influenced responses). Frequency-based detection techniques may examine the frequency spectrum of the muscle response to determine if the response has any fundamental frequency or harmonic correspondence to the stimulation frequency. Finally, pattern recognition techniques may analyze an individual sensor output, or the output from a grouping of sensors to recognize patterns that are indicative of an evoked/induced response.

If an evoked muscle response is detected, the GUI 102 may be altered, such as shown in FIG. 6, to reflect that the system has detected an evoked muscle response (i.e., one that is caused directly by the applied stimulus—also referred to as an artificially induced muscle response). For example, the textual status indicator 122 may reflect “Nerve Identified” and/or a visual indicator 124, such as the color of the background of the central status window 126, may be altered to reflect the detected evoked response. In one embodiment, where the “standby” state may be reflected by a first color (e.g., gray), and the “stimulating” state may be reflected by a second color (e.g., green), the “evoked response” state may be reflected, for example, by a third color (e.g., yellow).

As further illustrated in FIG. 6, the GUI 102 may also change the displayed channel to the channel that best evidences the induced muscle response. Because a single nerve root often innervates multiple muscles to varying degrees, if multiple muscle groups were to concurrently evidence an evoked response (i.e., by one or more of the techniques described above), the GUI 102 would, at a minimum, be altered to display the “primary muscle response.” In one configuration, the primary muscle response is the sensed response that has the maximum amplitude or magnitude. In another configuration, the primary muscle response would be the evoked muscle response that is the greatest when scaled relative to that muscle's supermaximal response (i.e., the measured response divided by supermaximal response), also referred to as the greatest scaled response.

In one configuration, the GUI 102 may further draw a visual connection between the sensor display element 104 representing the primary muscle response and the central status window 126. For example, in one configuration, the GUI 102 may represent a thick bar 150 between the primary sensor display element 152 and the central status window 126. This may provide a quick visual indication of which channel is being displayed and/or is primarily responding. Additionally, so that lesser responding channels are not simply ignored, a thinner bar 154 may join such ancillary channel sensor elements 156 (i.e., lesser relative response than the primary response) and the central status window 126. In one configuration, the thickness 158 of each bar may correspond to the relative magnitude of the response on that respective channel. It should be understood that the presently described visual techniques are illustrative of a manner of displaying the relative response intensities of different muscles that are artificially induced to respond. Other visual techniques may similarly be used without departing from the spirit of the present disclosure.

As further illustrated in FIG. 6, in some embodiments, a threshold 160 may be overlayed on the graph element 144. The threshold 160 may generally correspond to a physical distance (i.e., a “clearance distance”) that the user would like to maintain between the stimulator probe 40 and any nerve while performing the procedure. If the magnitude of the sensed response exceeds the threshold, the textual status indicator 122 may reflect “Alert” and/or a visual indicator 124, such as the color of the background of the central status window 126, may be altered to reflect the response exceeding the threshold. For example, the “Evoked Alert” state may be reflected, for example, by a fourth color (e.g., red).

While the user is making an approach toward a subject's spine, it is important to understand both when there is and when there isn't an evoked response. More specifically, the lack of a response (e.g., a green screen) will affirmatively convey a measure of safety to the user. Certain procedures, however, may involve techniques that are not always suitable for stimulation (thus limiting ability to sense an evoked response). These techniques may involve general gross manipulation of the subject, local manipulation of tissue, or other similar techniques such as muscle retraction. Such techniques, however, still pose some risks that a nerve will be stretched or strained in some way. To alert the user of the possibility of such an event, the system may be configured to monitor each sensor 34 for the occurrence of spontaneous MMG events. These are muscle events that are not caused by an affirmatively administered stimulus (e.g., an electrical stimulus), but rather by actions such as tissue manipulation or prolonged nerve compression.

Spontaneous MMG events may generally be detected by examining the MMG output signal such as described in US 2015/0051507, or in U.S. patent application Ser. No. 15/995,879.

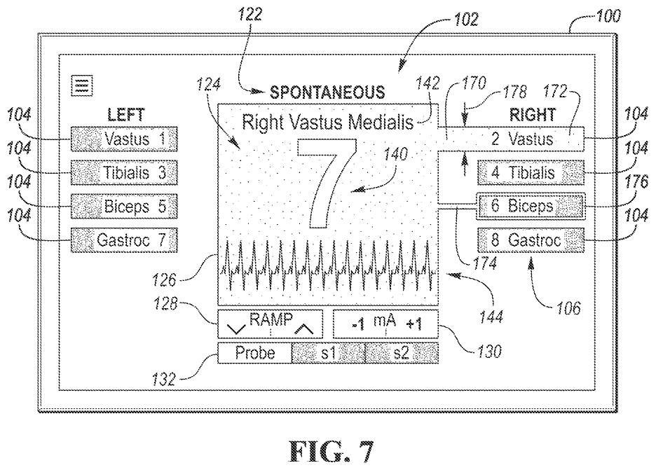

Similar to the evoked response, if a spontaneous muscle response is detected, the GUI 102 may be altered, such as shown in FIG. 7, to reflect that the system has detected a spontaneous muscle response. For example, the textual status indicator 122 may reflect “Spontaneous” and/or a visual indicator 124, such as the color of the background of the central status window 126, may be altered to reflect the detected spontaneous response. In one embodiment, the “spontaneous” state may be reflected, for example, by a fifth color (e.g., blue).

As further illustrated in FIG. 7, similar to the evoked response screen in FIG. 6, the GUI 102 may also change the displayed channel to the channel that detected the primary muscle response. Much like the evoked response, depending on the nerve that is manipulated, the response may be detected across multiple channels/sensors, with one likely being stronger than the other. As such, to highlight the primary response, the GUI 102 may draw a visual connection between the sensor display element 104 representing the primary spontaneous muscle response and the central status window 126. For example, in one configuration, the GUI 102 may represent a thick bar 170 between the primary sensor display element 172 and the central status window 126. Additionally, so that lesser responding channels are not simply ignored, a thinner bar 174 may join such ancillary channel sensor elements 176 (i.e., lesser relative response than the primary response) and the central status window 126. In one configuration, the thickness 178 of each bar may correspond to the relative magnitude of the response on that respective channel.

With reference to FIGS. 3 and 5-7, it should be appreciated that differing stipple patterns represent different colors. While the specific colors are not critical to the implementation of the present technology, it is important that a sufficient amount of contrast be maintained between the colors or imagery so that a user may be immediately alerted with a quickly recognizable and informative prompt. Ultimately, response time when dealing with relatively inelastic nerves or when detecting clearance distances on a millimeter scale is of the utmost importance. As such, the present disclosure provides a system that is capable of maximizing the quantity of information along with the readability of alerts.

While the best modes for carrying out the present technology have been described in detail, those familiar with the art to which this technology relates will recognize various alternative designs and embodiments that are within the scope of the appended claims. It is intended that all matter contained in the above description or shown in the accompanying drawings shall be interpreted as illustrative only and not as limiting.

1. A method comprising:
applying a plurality of mechanical sensors to a patient to detect muscle movement, each of the mechanical sensors producing an output signal on a corresponding sensor channel;
tapping a first sensor of the plurality of mechanical sensors; and
determining whether an output signal corresponding to the tap is received on a single sensor channel or on multiple sensor channels, wherein:
if the output signal is received on a single sensor channel, determining that the first sensor is operational and associating the first sensor and sensor channel to a muscle located adjacent to the first sensor; and
if the output signal is received on multiple sensor channels, determining that an error has occurred.

2. The method of claim 1, further comprising associating the single sensor channel of the first sensor to a first display element of a graphical user interface (GUI).

3. The method of claim 2, wherein first display element is labelled with the muscle associated with the single sensor channel.

4. The method of claim 1, further comprising tapping a second sensor of the plurality of mechanical sensors and determining whether an output signal corresponding to the tap is received on a single sensor channel or on multiple sensor channels.

5. The method of claim 4, wherein if the output signal is received on a single sensor channel, determining that the second sensor is operational and associating the second sensor and sensor channel to a muscle located adjacent to the second sensor.

6. The method of claim 5, further comprising associating the single sensor channel of the second sensor to a second display element.

7. The method of claim 4, further comprising tapping on each successive sensor of the plurality of mechanical sensors and determining, for each successive sensor, whether an output signal corresponding to the tap is received on a single sensor channel or on multiple sensor channels.

8. The method of claim 1, wherein the processor is further configured to display on a graphical user interface (GUI) any display elements representing sensors that are not operational with an indicia or a different color.

9. The method of claim 1, wherein each of the mechanical sensors converts a physical motion into the output signal.

10. The method of claim 9, wherein each of the mechanical sensors is a strain gauge, a pressure transducer, force transducer, a position encoder, an accelerometer, or a piezoelectric material.

11. An intraoperative neural monitoring system comprising:
a plurality of mechanical sensors to detect muscle movement, each of the mechanical sensors producing an output signal on a corresponding sensor channel; and
a processor configured to:
determine whether an output signal corresponding to a tap on a first sensor of the plurality of mechanical sensors is received on a single sensor channel or on multiple sensor channels;
if the output signal is received on a single sensor channel, determine that the first sensor is operational and associate the first sensor and sensor channel to a muscle located adjacent to the first sensor; and
if the output signal is received on multiple sensor channels, determine to display, on a display having a graphical user interface (GUI), an error message.

12. The system of claim 11, wherein the processor is further configured to associate the single sensor channel of the first sensor to a first display element of the GUI.

13. The system of claim 12, wherein first display element is labelled with the muscle associated with the single sensor channel.

14. The system of claim 11, wherein the processor is further configured to determine whether an output signal corresponding to a tap on a second sensor of the plurality of mechanical sensors is received on a single sensor channel or on multiple sensor channels.

15. The system of claim 14, wherein if the output signal is received on a single sensor channel, the processor is further configured to determine that the second sensor is operational and associate the second sensor and sensor channel to a muscle located adjacent to the second sensor.

16. The system of claim 15, wherein the processor is further configured to associate the single sensor channel of the second sensor to a second display element of the GUI.

17. The system of claim 14, wherein the processor is further configured to determine whether an output signal corresponding to a tap on each successive sensor of the plurality of mechanical sensors is received on a single sensor channel or on multiple sensor channels.

18. The system of claim 14, wherein the processor is further configured to display any display elements representing sensors that are not operational with an indicia or a different color.

19. The system of claim 11, wherein each of the mechanical sensors converts a physical motion into the output signal.

20. The system of claim 19, wherein each of the mechanical sensors is a strain gauge, a pressure transducer, force transducer, a position encoder, an accelerometer, or a piezoelectric material.
